(19) United States
(12) Patent Application Publication
(10) Pub. No.: US 2014/0249395 A1
(43) Pub. Date: Sep. 4, 2014

(54) Implantable Electrochemical Biosensors for Retinal Prostheses

(71) Applicant: Second Sight Medical Products, Inc., San Fernando, CA (US)

(72) Inventors: David Daomin Zhou, Canyon Country, CA (US); Robert J. Greenberg, Los Angeles, CA (US)

(21) Appl. No.: 14/195,608

(22) Filed: Mar. 3, 2014

Related U.S. Application Data
(60) Provisional application No. 61/771,788, filed on Mar. 1, 2013.

(51) Int. Cl. A61N 1/36; A61B 3/10; A61B 5/00

(52) U.S. Cl. CPC A61N 1/36046; A61B 5/6867; A61B 3/10
USPC 600/373; 607/54

(57) ABSTRACT

Progress has been made in the development of implantable electrochemical biosensors. However, to date a commercially-available long-term implantable biosensor is still out of reach. The foreign body response poses great challenges for long-term implantable devices. Retinal prostheses provide a platform for incorporation of biosensors for neural stimulation and biosensing in the human eye.

### FIELD

The present disclosure is generally directed to implantable biosensors, and more particularly to biosensors combined with neurostimulators such as visual prostheses.

### BACKGROUND

As intraocular surgical techniques have advanced, it has become possible to apply stimulation on small groups and even on individual retinal cells to generate focused phosphenes through devices implanted within the eye itself. This has sparked renewed interest in developing methods and apparatuses to aid the visually impaired. Specifically, great effort has been expended in the area of intraocular retinal prosthesis devices in an effort to restore vision in cases where blindness is caused by photoreceptor degenerative retinal diseases such as retinitis pigmentosa and age related macular degeneration which affect millions of people worldwide.

Neural tissue can be artificially stimulated and activated by prosthetic devices that pass pulses of electrical current through electrodes on such a device. The passage of current causes changes in electrical potentials across visual neuronal membranes, which can initiate visual neuron action potentials, which are the means of information transfer in the nervous system.

Based on this mechanism, it is possible to input information into the nervous system by coding the information as a sequence of electrical pulses which are relayed to the nervous system via the prosthetic device. In this way, it is possible to provide artificial sensations including vision.

One typical application of neural tissue stimulation is in the rehabilitation of the blind. Some forms of blindness involve selective loss of the light sensitive transducers of the retina. Other retinal neurons remain viable, however, and may be activated in the manner described above by placement of a prosthetic electrode device on the inner (toward the vitreous) retinal surface (epiretinal). This placement must be mechanically stable, minimize the distance between the device electrodes and the visual neurons, and avoid undue compression of the visual neurons.

In 1986, Bullara (U.S. Pat. No. 4,573,481) patented an electrode assembly for surgical implantation on a nerve. The matrix was silicone with embedded iridium electrodes. The assembly fit around a nerve to stimulate it.

Dawson and Radtke stimulated cat's retina by direct electrical stimulation of the retinal ganglion cell layer. These experimenters placed nine and then fourteen electrodes upon the inner retinal layer (i.e., primarily the ganglion cell layer) of two cats. Their experiments suggested that electrical stimulation of the retina with 30 to 100 uA current resulted in visual cortical responses. These experiments were carried out with needle-shaped electrodes that penetrated the surface of the retina (see also U.S. Pat. No. 4,628,933 to Michelson).

The Michelson '933 apparatus includes an array of photosensitive devices on its surface that are connected to a plurality of electrodes positioned on the opposite surface of the device to stimulate the retina. These electrodes are disposed to form an array similar to a “bed of nails” having conductors which impinge directly on the retina to stimulate the retinal cells. U.S. Pat. No. 4,837,049 to Byers describes spike electrodes for neural stimulation. Each spike electrode pierces neural tissue for better electrical contact. U.S. Pat. No. 5,215,088 to Norman describes an array of spike electrodes for cortical stimulation. Each spike pierces cortical tissue for better electrical contact.

The art of implanting an intraocular prosthetic device to electrically stimulate the retina was advanced with the introduction of retinal tacks in retinal surgery. De Juan, et al. at Duke University Eye Center inserted retinal tacks into retinas in an effort to reattach retinas that had detached from the underlying choroid, which is the source of blood supply for the outer retina and thus the photoreceptors. See, e.g., de Juan, et al., 99 Am. J. Ophthalmol 272 (1985). These retinal tacks have proved to be biocompatible and remain embedded in the retina, with the choroid/sclera, effectively pinning the retina against the choroid and the posterior aspects of the globe. Retinal tacks are one way to attach a retinal array to the retina. U.S. Pat. No. 5,109,844 to de Juan describes a flat electrode array placed against the retina for visual stimulation. U.S. Pat. No. 5,935,155 to Humayun describes a retinal prosthesis for use with the flat retinal array described in de Juan.

Implantable neural stimulators, such as visual prostheses, must be inductively link to an outside source of power and data. The better the link between the two coils, the less power is required to operate the neural stimulator. Since the internal coil is fixed at the time of implantation, all adjustment must be made to the external coil. Systems are needed to quickly identify the optimal position for the external coil and to hold the external coil in that position for use.

### BRIEF DESCRIPTION OF THE DRAWINGS

FIG. 1 is a schematic of approaches of retinal prostheses in the human eye.

FIG. 2 shows a perspective view of the implantable portion of the visual prosthesis.

FIG. 3 is a side view of the implantable portion of the visual prosthesis.

FIG. 4 shows an arrangement comprising a visor, a visual processing unit and a cable connecting the visor to the visual processing unit.

### DETAILED DESCRIPTION

Progress has been made in the development of implantable electrochemical biosensors. The foreign body response poses great challenges for long-term implantable devices. Retinal prostheses provide a platform for incorporation of biosensors for neural stimulation and biosensing in the human eye. Blindness has a devastating impact on people's quality of life, and it can result from diseases or injuries to any part of the visual pathway. Visual pathway consists mainly of the eye, optic nerve, lateral geniculate nucleus (LGN) and visual cortex (also known as striate cortex or VI). When the light reaches the retina through the cornea and the pupil, photoreceptors on the outer boundary layer of the retina membrane convert photons into electrical neural signals. These signals are processed by cells in the retina structure, sent to the brain along the optic nerves and perceived as visual percepts. Research efforts worldwide are developing microelectronic visual prostheses using electrical stimulation aimed at restoring vision for the blind.

### Retinal Stimulation and Retinal Prostheses

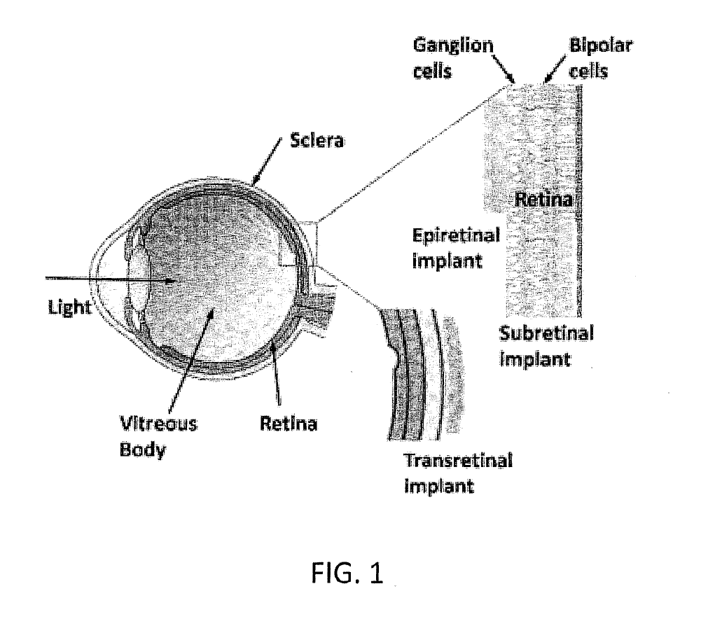

The greatest progress toward artificial vision to date has been in the development of retinal prostheses. Retinal prostheses, both epiretinal and subretinal have been shown to partially restore visual function to patients blinded by retinal degenerative diseases, such as retinitis pigmentosa. Retinitis pigmentosa (RP) is a group of inherited diseases that destroy the photoreceptor cells located in the retina. People with RP experience a gradual decline in their vision and eventually become blind because of loss of photoreceptors. In spite of nearly complete degeneration of the retinal architecture there is relative preservation of the inner retinal neurons. The approach of retinal stimulation by an intraocular prosthesis is to electrically stimulate the remaining retinal cells, bypassing the degenerated photoreceptors. Epiretinal approaches involve placing electrodes on the top side of the retina near ganglion cells, whereas subretinal approaches involve placing electrodes and most of the electronics under the retina in the location of the degenerated photoreceptors between the retina and the retinal pigment epithelium (FIG. 1). Even more recently, placement of the electrode outside the eye or between the sclera and choroid has also been proposed

### The Argus II Retinal Prosthesis System

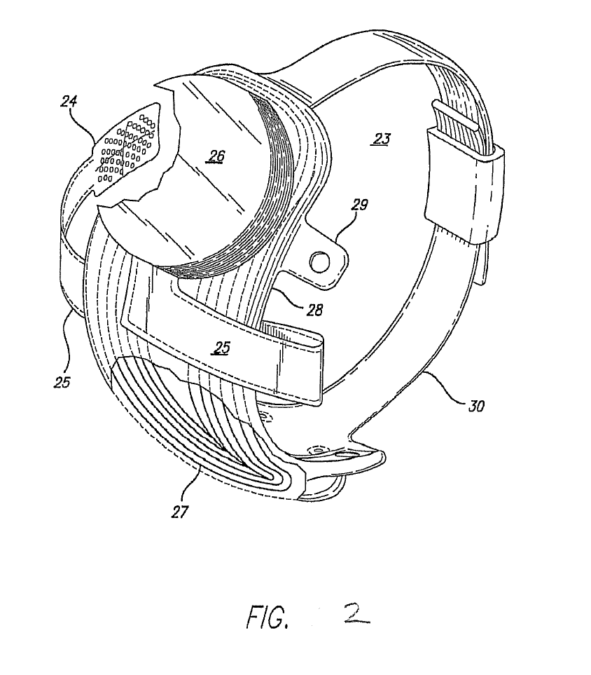

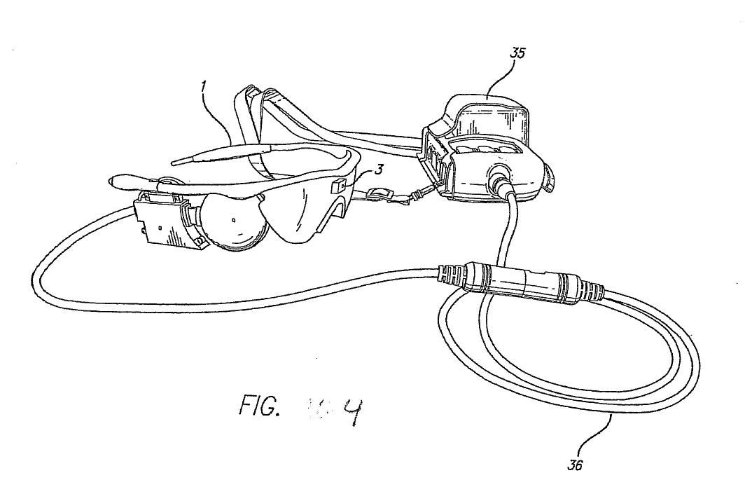

The Argus II retinal prosthesis system consists of implanted and external components. The implant is an epiretinal prosthesis that includes a receiver antenna, electronics, and an electrode array (FIG. 2). The flexible thin-film polymer array has 60 platinum electrodes arranged in a 6×10 grid that are attached to the epiretinal surface over the macula with a retinal tack. The external equipment includes glasses, a video processing unit (VPU) and a cable (FIG. 4). The glasses include a miniature video camera, which captures video images, and a coil that transmits data and stimulation commands to the implant. The VPU converts the video images into stimulation commands and is body-worn. A cable connects the glasses to the VPU. The Argus II system operates by converting video images into electrical pulses that activate retinal cells, delivering the signal through the optic nerve to the brain where it is perceived as light. The Argus II retinal prosthesis system is the first commercially-available CE-marked and Food and Drug Administration (FDA) approved treatment for RP.

### Biosensing in the Human Eye

The eyeball is slightly ellipsoidal and has a volume of about 10 cm3 in an adult 18-30 years of age. The axial length is approximately 24 mm from the cornea to the retina. The human retina that lines the back of the eye is approximately 250 μm thick, and is a delicate multilayered organization of neurons, cells and nourishing blood vessels. The space inside the eye has a volume of about 4-6.5 ml and is filled with clear vitreous humor. Table 1 lists the concentrations of some biochemicals in the vitreous humor. The vitreous is a gel that consists of collagen fibers that are separated and stabilized by hyaluronic acid. Approximately 98% of this gel is water; diffusion of low molecular-weight solutes such as inorganic ions, glucose and amino acids is unimpeded through the vitreous. Chronic implantable retinal prostheses or other similarly sized electronic implants with communication links to outside the body using technologies developed for retinal prostheses can provide a platform for biosensing in the human eye.

Table 1. The Concentrations of Some Chemicals in the Vitreous Humor.

Chemical Concentration μM

Ascorbate 2.21

Lactate 7.78

Glucose 3.44

Pyruvate 0.81

Collagen 286 μg/ml

L-Glutamate˜0.1-10

The major substrate for respiration in the retina is glucose. Most of the glucose (˜70%) utilized by the retina is converted to lactate. The nutritional supplies for the retina, including glucose, are provided by both choroidal and retinal circulation. An exceptionally high rate of glucose metabolism inside the retina may be the cause for lower glucose concentration in vitreous humor than that in plasma. Clinical studies suggest that chronic implantation of subretinal arrays likely obstructed the nourishment to the retina and caused both inner and outer retina damage. Closely monitoring the glucose concentration changes during retinal stimulation and array implantation could reveal such blockage of nourishment. Glutamate is a neurotransmitter in the retina, and has been found in higher concentration within the retina. Studies on neural stimulation have established the link between the activation of neurons and the release of glutamate. High levels of glutamate have demonstrated neurotoxicity. Glutamate is actively metabolized by normal retina tissue and has served as an indicator of glaucoma and diabetic retinopathy. Sensing glucose and glutamate in the human eye would be of clinical importance.

### Electrochemical Glucose Biosensors

Electrochemical detection principle is compatible with electrical stimulation based implants which control either current or voltage of implantable electrodes. Enzyme electrode based electrochemical biosensors comprise the most extensively studied class of biosensors. Amperometric biosensors are based on the oxidation/reduction of electro active species generated or consumed in an enzymic reaction. Typical amperometric glucose biosensors consist of a thin layer of glucose oxidase (GOx) entrapped or immobilized over an oxygen or hydrogen peroxide electrode via a semipermeable dialysis membrane. Amperometric measurements are made based on monitoring the oxygen consumed or the hydrogen peroxide generated by the enzyme-catalyzed reaction:

D-glucose+O₂+H₂O+GOx→gluconic acid+H₂O₂ (1)

An anodic potential is applied to the platinum working electrode to detect hydrogen peroxide via an oxidation reaction:

H₂O₂→2H⁺=O₂+2e⁻ (2)

A cathodic potential is applied to the platinum working electrode to detect oxygen consumption via a reduction reaction:

O₂+4H+4e⁻→2H₂O (3)

### Subcutaneous Continuous Glucose Sensors

Currently commercial implantable glucose biosensors are limited to subcutaneous continuous glucose sensors for managing diabetes. Minimally invasive subcutaneous sensors measure the interstitial glucose concentration through continuous measurement of interstitial fluid (ISF) rather than that of blood. Three FDA approved continuous glucose monitoring (CGM) systems are commercially available. Medtronic MiniMed's (Northridge, Calif.) subcutaneous needle electrodes measure glucose by an amperometric method based on a glucose oxidase and hydrogen peroxide system. Interstitial glucose is converted by the glucose oxidase to produce hydrogen peroxide, which is oxidized on a platinum electrode to generate an amperometric response. The sensor has a lifetime of 3 days, measures interstitial glucose every 10 seconds, and reports an average glucose concentration every 5 minutes. Dexcom's Seven Plus (San Diego, Calif.) also utilizes a subcutaneous continuous glucose sensor. The sensor has a 7 day lifetime. Abbott's Freestyle Navigator (Alameda, Calif.), discontinued in the U.S since late 2011, had a 5 day lifetime.

### Chronic Implantable Biosensors

A long-term implantable glucose sensor developed by DexCom was reported in 2004 [II]. The sensor was implanted in the subcutaneous tissue of the abdomen in 15 patients with type 1 diabetes. The sensor was about the size and shape of an AA battery. Its bulk size may prevent DexCom's implantable sensor from practical application and it is thus far not commercially available. Recently, a miniature fully implantable continuous glucose sensor has been reported. The sensor, developed by David Gough and Glysens (San Diego, Calif.) is capable of long-term monitoring of tissue glucose concentrations while implanted in subcutaneous tissues of two pigs for more than one year. Sensor arrays were fabricated by a patterned thick film of platinum paste co-fired with an alumina disc substrate, then brazed to a titanium case to form a hermetic package. The electrochemical detection of glucose, in a three electrode-mode by a battery powered potentiostat, uses two differential platinum oxygen working electrodes, a platinum counter electrode and an Ag/AgCI reference electrode. The electrodes are covered by a thin electrolyte layer, a protective layer of oxygen permeable polydimethylsiloxane (PDMS), and a membrane of PDMS with wells for the immobilized enzymes located over certain electrodes. The large reserve of enzymes is immobilized in the wells by cross-linking with albumin using glutaraldehyde. An interesting feature in the sensor design is to use excess co-immobilized catalase to convert hydrogen peroxide to oxygen in preventing peroxide induced enzyme inactivation and possible tissue irritation. In a three-electrode mode, a potentiostat applies a potential on the working electrode against a reference electrode while the response current is measured between the working and a counter electrode. There is little or, ideally, no current pass through the reference electrode to cause polarization, thus a stable potential will be maintained. The counter electrode has a considerably larger area than that of the working electrode to minimize any electrochemical reaction over its surface, so that the response current measured is dominated by the desired reaction on the working electrode.

### Electrochemical Glutamate Biosensors

Most amperometric glutamate biosensors are operated in a way similar to the glucose biosensors. L-glutamate oxidase (GluOx) is chemically immobilized in a membrane which is in contact on one side with the sample solution and on the other with the electrode. L-glutamate diffuses into the enzyme layer where it is oxidized by the enzyme according to the reaction: 4

L-glutamate+O2+H₂O+GluOx->α-ketoglutarate+H₂O₂+NH₃ (4)

Similar to the glucose sensing, either the decrease in oxygen or the increase in hydrogen peroxide at the electrode surface is measured and they are directly proportional to the L-glutamate concentration in the sample solution.

Currently there are no implantable commercial glutamate biosensors available. Most work on glutamate sensors remains in R&D stages. Silicon wafer-based platinum micro electrode arrays modified with glutamate oxidase, polypyrrole and Nafion® are used for detection of electrical stimulation-evoked glutamate release in the ventral striatum of the ambulant rat. An iridium oxide reference electrode is incorporated on the microelectrode array to replace the commonly used Ag/AgCI electrode. An implantable L-glutamate sensor array on a flexible polyimide substrate has been reported. L-glutamate oxidase is immobilized on the electrode with bovine serum albumin and glutaraldehyde, then protected by a layer of electro-polymized meta-phenylenediamine. The array is capable of sensing neurotransmitters and recording extracellular action potentials simultaneously.

### Challenges in the Development of Implantable Biosensors

While many challenges exist in the development of implantable sensors, the foreign body response (FBR) induced challenges, such as implant packaging, chronic stability, and in-vivo calibration of implantable sensors, are of paramount importance. The foreign body response (FBR) initiated by the implantation of medical devices includes a certain sequence of biological events: injury, inflammatory cell infiltration, acute inflammation, chronic inflammation, granulation tissue formation, the foreign body reaction, and eventual fibrosis/fibrous encapsulation.

### Hermetic Packages of Implants

Packaging of implanted medical devices is one of the greatest challenges in the biomedical industry. Three approaches have been pursued in the active implant packages: hard-cases including metal, ceramic or glass cases, soft-cases including various polymer encapsulations and thin-film chip-scale packages (CSP). The hard-case approach has been used exclusively by various implantable device manufacturers. Titanium appears to be the material of choice for the hard case packages. Soft-case materials include silicone (PDMS and its derivatives), epoxies, and various polymers such as parylene, polyurethane and polyimide. Thin-film chip scale package technology, developed for semiconductor industries, will potentially result in a slim hermetic package that is virtually the same size as the bare stimulator chip. Thin-film CSP coating materials include silicon oxide, silicon nitride, silicon carbide, alumina, diamond-like carbon, polycrystalline diamond, and ultra-nano-crystalline diamond (UNCD).

### Chronic Stability of Implantable Sensors

In contrast to the excellent stability and sensitivity of most sensors' function in-vitro, a reduction in sensitivity occurs after implantation, with a resulting rapid decrease in-vivo signal followed by complete loss of sensor function within hours or days. The FBR induced sensor encapsulation and interferences due to biofouling of electrode, and enzyme activity loss seems to be major causes. Interferences of biochemical and electrochemical origins affect an implantable sensors performance. In biochemical interference such as enzymatic interference, inhibitors, activators or non-specific and impure enzymes will affect enzyme activities. In electrochemical interference, any species available on electrode surface which are electroactive over the same potential range as the analyte will interfere with the measurement.

Using biocompatible materials in implant packaging to minimize the FBR is critical. In addition, the mechanical aspects of implants, such as size, shape, material modulus, and texture, need to be considered. Effects of implant motion, pressure-induced interfacial stress and implant design affect biomechanics of the sensor-tissue interface and alter the FBR toward the implants.

The biofouling of the electrode surface by adsorption of protein can be minimized by including a separation or blocking membrane. The thickness and pore size are two key factors for the membrane selectivity. Cellulose acetate (CA), a selectively permeable membrane, is widely used as the blocking membrane. The CA membrane's molecular weight cut-off of 100 daltons will effectively exclude proteins but not oxygen and hydrogen peroxide. CA membranes are also capable of retarding the transport of anionic species such as ascorbate and urate, two major interferents, particularly when hydrogen peroxide is monitored. A novel approach may have application in minimizing biofouling by using a self-cleaning membrane.

Electrochemical interference can be minimized by lowering the oxidation or reduction potentials using highly stable and electro catalytic electrode materials, such as high surface platinum gray, some novel conducting polymers and many nanomaterials. Platinum gray is described in U.S. Pat. No. 6,974,533, which is incorporated herein by reference. Using a mediator (Mediators are small molecules and electroactive redox couples, either bound to the electrode surface or free in solution, able to shunt electrons between an electrode and an analyte) to reduce the oxidation potential is also a possible method to reduce electrochemical interference. Osmium complexes and ferrocene and its derivatives are some important mediators. Using a dual working electrode design can also mitigate the interference. A blank working electrode is prepared the same as the main working electrode, except no enzyme is on the electrode surface. The background signal is measured by this blank electrode and subtracted by the signal from the main working electrode.

In a long-term implanted application, a sufficient reserve of enzymes must be contained in a sensor or be ready to be replenished. The stability of such stored enzymes also greatly affects the life-time of the implantable biosensors. Using electrochemical non-enzymatic glucose sensors has been explored to avoid this problem. Non-enzymatic glucose detection involves the direct electro-oxidation of glucose to gluconic acid. Glucose oxidation is a kinetically very slow process which requires electrodes to have high electrocatalytic activity. Nanostructured platinum, palladium and gold electrodes with very high surface areas and electro catalytic activity are promising for the direct glucose oxidation. The slow kinetic process and lack of specificity due to interferences from other sugars are two major hurdles for the direct electro-oxidation approach.

### Oxygen Deficit Issue

The perturbation of the co-substrate or analyte concentration is another hurdle for the implantable sensors in continuous biosensor operation. Oxygen concentration variation (as a co-substrate or as an analyte) and the low ratio of oxygen to glucose that exists in the body pose a challenge in oxidase based glucose biosensors which are sensitive to the variation in oxygen content. One way to tackle this problem is to use a glucose dehydrogenase enzyme for the conversion of glucose to measurable redox equivalents. Alternatively, a two dimensional diffusion mechanism can be used to tailor the glucose/oxygen permeability ratio in promoting oxygen diffusion. The sensor design with a cylinder oxygen collection tube containing GOx/gel is made of silicone which is impermeable to glucose but highly permeable to oxygen. This allows oxygen to diffuse into the gel layer of the sensor through the large silicone tubing's side wall and the exposed end, but allows glucose to only diffuse through the small cross-section of the exposed end.

### In-Vivo Calibration of Implantable Sensors

For an implantable sensor, the background current in-vivo is likely to be higher than in-vitro due to current produced by electrochemical interferents. When the bioagent's activity is lowered, the sensor's response will be changed too. How to calibrate the device in-vivo becomes a significant problem. In practice, a one-point or two-point calibration process is used to “update” the sensor's calibration curve. Two-point calibration requires two substrate readings to determine both the slope (sensitivity) and the intercept (background current) of the sensor's response. One-point calibration takes one reading and assumes the intercept is zero or uses a non-enzyme electrode to measure the background signal. In most implantable biosensors such as MiniMed's CGM sensor systems, a commercial glucose meter is used to calibrate the implanted sensor. In continuously operated glucose sensors, a time-lag, which is associated to the sensor design, the substrate mass transfer rate in tissue and the dynamic rate of glucose change in the body, exists between blood glucose and interstitial glucose in tissue. Such time differences should be taken into account in the recalibration of sensors.

Toward Retinal Prostheses or Electronic Ocular Implants with Multi-Analyte Biosensors

The success in chronic retinal prostheses and advance in biosensor research bring hope for a fully implantable biosensing system by combining the technologies developed for retinal prostheses with those for multi-analyte biosensors. The retinal prostheses' high density thin-film electrode array provides a platform to the integration of multi-analyte biosensing elements (FIG. 2). The multiple-analyte sensing approach has been proven feasible in a study in which glucose, lactate, glutamate and ATP were monitored simultaneously using a battery powered implantable carbon nanotude sensor in mice. Multiple potentiostats for amperometric measurements can be integrated into the retinal implant's ASIC chip for multi-analyte detection. Unlike most fully implantable biosensors which contain bulky primary batteries, the retinal implant does not require a battery. Both data and electrical power are wirelessly transmitted between an external receiver and the implant, thus resulting in a miniature device. In addition to biosensors, chemical sensors and drug delivery elements can be also integrated to enhance the device function. Using novel nanotechnologies combined with well established MEMs methods will produce reliable electrodes for neural stimulation and for real-time biosensing inside the eye.

### CONCLUSION

Advances in biomedical engineering, microfabrication technology, neuroscience and biosensor technology will accelerate the research and development efforts from both academics and industries toward a fully implantable long-term functional biosensor system. Retinal prostheses restore partial vision to patients with retinitis pigmentosa. The implants work by electrically stimulating the remaining retinal cells, bypassing the degenerated photoreceptors. Biosensing in the human eye has clinical importance and retinal prostheses provide a platform for incorporation of electrochemical biosensors, thus allowing simultaneous neural stimulation and biosensing. Currently, commercially available implantable biosensors are subcutaneous sensors for short-term (3-7 days) continuous glucose monitoring. Progress in the development of long-term fully implantable biosensors has been made. However, many scientific and engineering challenges remain. An interdisciplinary effort and collaboration between academic institutes and biomedical industries, such as the collaboration that developed the Argus II, is essential for the successful development of fully implantable biosensor systems.

FIG. 2 shows a perspective view of an implantable portion 23 of a retinal prosthesis as disclosed. An electrode array 24 is mounted by a retinal tack or similar means to the epiretinal surface. The electrode array 24 is electrically coupled by a cable 25, which can pierce the sclera and be electrically coupled to an electronics package 26 external to the sclera. Electronic package 26 includes the RF receiver and electrode drivers.

The electronics package 26 can be electrically coupled to the secondary inductive coil 27. In one aspect, the secondary inductive coil 27 is made from wound wire. Alternatively, the secondary inductive coil may be made from a thin film polymer sandwich with wire traces deposited between layers of thin film polymer. The electronics package 26 and secondary inductive coil 27 are held together by a molded body 28. The molded body 28 may also include suture tabs 29. The molded body narrows to form a strap 30 which surrounds the sclera and holds the molded body 28, secondary inductive coil 27, and electronics package 26 in place. The molded body 28, suture tabs 29 and strap 30 are preferably an integrated unit made of silicone elastomer. Silicone elastomer can be formed in a pre-curved shape to match the curvature of a typical sclera. Furthermore, silicone remains flexible enough to accommodate implantation and to adapt to variations in the curvature of an individual sclera. In one aspect, the secondary inductive coil 27 and molded body 28 are oval shaped, and in this way, a strap 30 can better support the oval shaped coil.

The entire implantable portion 23 is attached to and supported by the sclera of a subject. The eye moves constantly. The eye moves to scan a scene and also has a jitter motion to prevent image stabilization. Even though such motion is useless in the blind, it often continues long after a person has lost their sight. By placing the device under the rectus muscles with the electronics package in an area of fatty tissue between the rectus muscles, eye motion does not cause any flexing which might fatigue, and eventually damage, the device.

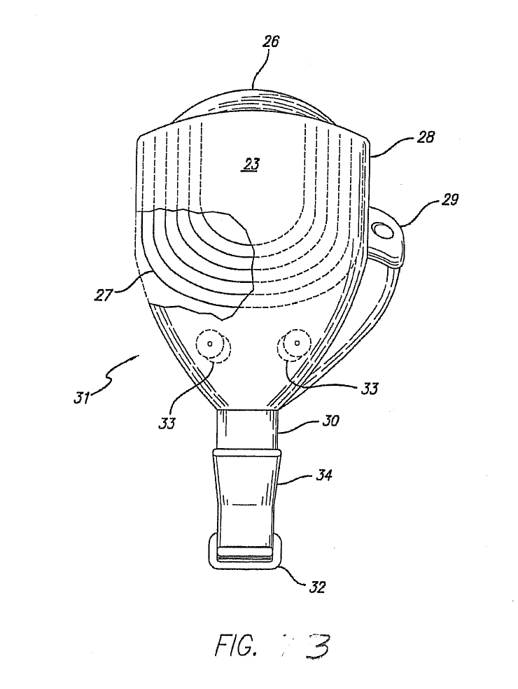

FIG. 3 shows a side view of the implantable portion of the retinal prosthesis, in particular, emphasizing the fan tail 31. When the retinal prosthesis is implanted, the strap 30 has to be passed under the eye muscles to surround the sclera. The secondary inductive coil 27 and molded body 28 should also follow the strap under the lateral rectus muscle on the side of the sclera. The implantable portion 23 of the retinal prosthesis is very delicate. It is easy to tear the molded body 28 or break wires in the secondary inductive coil 27. In order to allow the molded body 28 to slide smoothly under the lateral rectus muscle, the molded body is shaped in the form of a fan tail 31 on the end opposite the electronics package 26. Element 32 shows a retention sleeve, while elements 33 and 34 show holes for surgical positioning and a ramp for surgical positioning, respectively.

FIG. 4 shows an arrangement comprising a visor, a visual processing unit and a cable connecting the visor to the visual processing unit. This is as the device is used in stand-alone mode. The signal strength indicator would not be connected to the visual prosthesis as used by a patient. The visor 1, as described above is connected by a cable 36 to a video processing unit 35 as worn by the patient.

In summary, new technologies developed for electrical visual stimulation can be adapted to create a new biosensor. The apparatus provides a means for electrically sensing chemical present in the body. While the invention has been described by means of specific embodiments and applications thereof, it is understood that numerous modifications and variations could be made thereto by those skilled in the art without departing from the spirit and scope of the invention. It is therefore to be understood that within the scope of the claims, the invention may be practiced otherwise than as specifically described herein.

What is claimed is:

1. An implantable biosensor comprising:
a sensing element;
an electronic circuit receiving signals from the sensing element and producing sensing data;
an implantable hermetic package incasing the electronics circuit; and
a coil for sending the sensing data from the biosensor outside the body;
wherein the biosensor is of suitable size and geometry for implantation in and around an eye.

2. The implantable biosensor according to claim 1, wherein the sensing element includes at least three electrodes.

3. The implantable biosensor according to claim 2, wherein at least one of the electrodes is a high surface area noble metal.

4. The implantable biosensor according to claim 3, wherein the high surface area noble metal is platinum gray.

5. The implantable biosensor according to claim 2, further comprising a mediator on at least one of the electrodes.

6. The implantable biosensor according to claim 5, wherein the mediator comprises Osmium.

7. The implantable biosensor according to claim 5, wherein the mediator comprises ferrocene.

8. The implantable biosensor according to claim 1, wherein the sensing element senses glucose oxidation.

9. The implantable biosensor according to claim 1, wherein the sensing element is suitable to be placed outside the eye.

10. An implantable visual prosthesis and biosensor comprising:
a thin film electrode array sensing element for stimulating visual neurons and sensing biochemistry;
an electronic circuit receiving signals from the sensing element and producing sensing data and receiving stimulation signals and driving stimulation signals on the thin film electrode array;
an implantable hermetic package incasing the electronics circuit; and
a coil for sending the sensing data from the biosensor outside the body and receiving stimulation data from outside the body;
wherein the visual prosthesis and biosensor is of suitable size and geometry for implantation in and around an eye.

11. The implantable visual prosthesis and biosensor according to claim 10, wherein at least one of the electrodes is a high surface area noble metal.

12. The implantable visual prosthesis and biosensor according to claim 11, wherein the high surface area noble metal is platinum gray.

13. The implantable visual prosthesis and biosensor according to claim 10, further comprising a mediator on at least one of the electrodes.

14. The implantable visual prosthesis and biosensor according to claim 13, wherein the mediator comprises Osmium.

15. The implantable visual prosthesis and biosensor according to claim 13, wherein the mediator comprises ferrocene.

16. The implantable visual prosthesis and biosensor according to claim 10, wherein the sensing element senses glucose oxidation.

17. The implantable visual prosthesis and biosensor according to claim 10, wherein the flexible circuit electrode array is an epiretinal array.

18. The implantable visual prosthesis and biosensor according to claim 10, wherein the implantable hermetic package is attached to a sclera.

19. The implantable visual prosthesis and biosensor according to claim 10, wherein the sensing element is suitable to be implanted in a subretinal location.

20. The implantable visual prosthesis and biosensor according to claim 10, wherein the sensing element is suitable to be implanted in a superchoroidal location.
